(19) United States
(12) Patent Application Publication
(10) Pub. No.: US 2012/0010227 A1
(43) Pub. Date: Jan. 12, 2012

(54) FORMULATION FOR NAIL AND NAIL BED DISEASES

(75) Inventors: Lusiana, Braunschweig (DE); Christel Müller-Goymann, Braunschweig (DE)

(73) Assignee: Technische Universitat Braunschweig, Braunschweig (DE)

(21) Appl. No.: 12/846,536

(22) Filed: Jul. 29, 2010

(30) Foreign Application Priority Data
Jul. 30, 2009 (DE) DE 102009035589.8
Aug. 25, 2009 (DE) DE 102009038512.6

(51) Int. Cl. A61K 31/513; A61P 31/10; A61K 31/343; A61K 31/4412; A61K 31/137; A61K 31/351

(52) U.S. Cl. 514/274; 514/655; 514/459; 514/462; 514/345

(57) ABSTRACT

The invention relates to a pharmaceutical composition with a content in active ingredient that can be an antimycotic, so that the composition is suitable for the treatment of onychomycoses, in particular severe onychomycoses. The inventive formulation is an ointment-like thermogel at body temperature and an inviscid thermogel at lower temperatures and facilitates the permeation of sufficient active ingredient quantities through human fingernails and toenails, in order to also treat severe onychomycoses.

### PRIORITY CLAIM AND REFERENCE TO RELATED APPLICATION

The application claims priority under 35 U.S.C. §119 from prior German application Nos DE 10 2009 035 589.8, which was filed on Jul. 30, 2009, and DE 10 2009 038 512.6, which was filed on Aug. 25, 2009.

### FIELD

A field of the invention is formulations for nail and nail bed diseases.

### BACKGROUND

EP 1390031 B1 discloses pharmaceutical compositions for topical application containing a combination of terbinafine with a second antimycotic, and which for example can be formulated as an emulsion. The aqueous phase can contain C1 through C4 alcohols, polyhydroxy-C2-C5-alkanes, poly-C2-C5 alkylene glycols and a water-soluble non-ionic tenside as solvents, however no anionic tenside. Surface mycoses caused by dermatophytes are named as indications for this formulation. In addition with regard to the formulation it is specified that the active ingredient combination can also be provided as a nail lacquer. A formulation for a corresponding nail lacquer is not specified.

EP 1207861 B1 describes antifungicidal compositions containing nutrients for the infectious fungi along with an antimycotic. Fungal infections of the nail (onychomycoses) are also named as an indication.

EP 0503988 B1 describes a pharmaceutical composition for use against onychomycoses containing terbinafine hydrochloride as an active ingredient in an aqueous formulation with a C2-C8-Alkanol and a penetration reinforcing agent which is selected from glycols, glycol monoethers, glycol diethane, dimethyl sulfoxide, caprolactam, dimethyl isosorbide, isopropylidene glycerol, dimethylimidazolidinone, 2-methylpyrrolidone-2-pyrrolidone-2, ethyl lactate, polyoxyethylated C8-10-glycerides, polyethylene glycole-20-glycerol laureate, and dimethyl acetamide.

EP 0247142 B1 describes film-forming formulations for antimycotics for application in the case of onychomycoses. The formulations contain aqueous mixtures of cellulose resins, polyethylene oxide resins, acrylic acid and acrylate resins, vinyl resins and polyvinyl pyrollidone. It is specified that the compositions dry on the nail within circa 15 minutes.

WO 99/53913 describes a plurality of gel compositions for antimycotics for exterior application. Terbinafine is not named among the cited fungicides.

Winkler and Müller-Goymann (European Journal of Pharmaceutics and Biopharmaceutics 427-437 (2005)) describe among other things a formulation which contains 1 percent by weight medium chain triglycerides, 26 percent by weight water, 8% lutrol F 127 (poloxamer 407), 5 percent by weight dimethyl isosorbide, 8 percent by weight isopropanol in mixture with 2.5 percent by weight ibuprofen acid, as well as two similar formulations in which however either the medium chain triglycerides or medium chain triglycerides and ibuprofen acid are lacking, in each case for permeation reinforcement of 5-aminolevulinic acid for photodynamic therapy, e.g. against for skin cancer.

DE 10 2005 019628 describes formulations which reinforce the permeation of among other things clotrimazole and terbinafine through the human skin, and contain 10-30 percent by weight polyether polyol, 2.5-10 percent by weight lipids, 5-20 percent by weight dimethyl isosorbide, 5-20 percent by weight C2-C4-alcohol and 40-60 percent by weight water.

### SUMMARY OF THE INVENTION

The invention solves the problem with the features in accordance with the claims and in particular provides a formulation for use as a medicament for the treatment of nail and/or nail bed diseases, e.g., of nail mycoses (onychomycoses), said formulation containing a pharmaceutical active ingredient in a formulation on an aqueous basis or consisting of at least one active ingredient and the formulation which contains or consists of:

0, 1-20 percent by weight at least of an apolar, lipophilic, amphiphilic, zwitterionic or strongly polar, pharmaceutically acceptable active ingredient, preferably terbinafine, in particular terbinafine hydrochloride,

10-50 percent by weight, preferably 10-30 percent by weight of a polyetherpolyol or a mixture thereof, for example selected from polyethylene glycol (α-hydro-ω-hydroxy-poly(oxy-1,2-ethane diol)), polypropylene glycole and/or poloxamer (especially preferably poloxamer 407, α-hydro-ω-hydroxy-poly(oxyethylene)-poly(oxypropylene)-poly(polyoxyethylene)-block copolymer,

5-25 percent by weight dimethyl isosorbide,

2.5-10 percent by weight triglycerides, preferably medium-chain triglycerides, in particular according to the German Pharmacopoeia, and

5-20 percent by weight of a C₁- to C₄-alcohol or a mixture thereof, preferably selected from ethanol and isopropanol,

remainder water,

preferably without ibuprofen,

especially for the treatment using tropical application onto the nail, and optionally additionally containing a pharmaceutical by acceptable colorant or perfume

### BRIEF DESCRIPTION OF THE DRAWING

The invention will now be described more closely on the basis of examples with reference to the figure, which shows

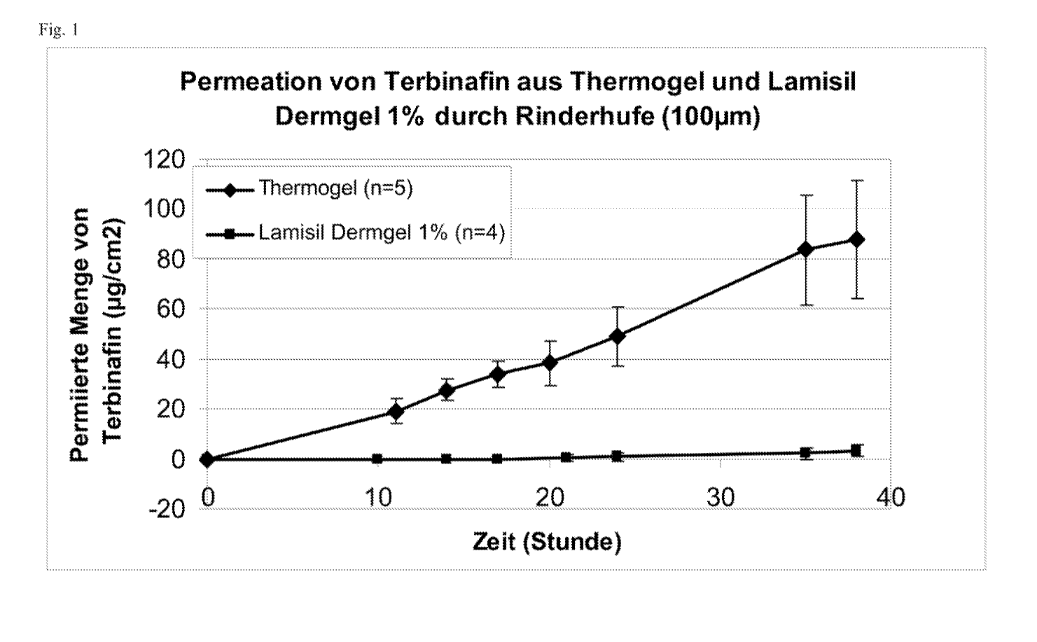

in FIG. 1 the graphic evaluation of the quantity of terbinafine hydrochloride permeated through a bovine hoof disc for an inventive formulation (♦) and for Lamisil Dermgel 1% (▪).

### DETAILED DESCRIPTION OF THE PREFERRED EMBODIMENTS

The invention relates to a pharmaceutical composition with a pharmaceutical active ingredient content which is characterized by the special promotion of the penetration and permeation of the active ingredient into and through fingernails and toenails, in particular in the case of topical application on nail. A polar, amphiphilic or lipophilic active ingredient can be contained as the pharmacentical active ingredient in the composition, e.g., dithranol, preferably in combination with urea, or vitamin D preparations, e.g., calcipotriol and/or tacalcitol, or corticoid, e.g., betamethasone, hydrocortisone, clobetasol, triamcinolonacetonide, prednicarbate and/or fluticasone, or retinoids, e.g., adapalene, tazarotene, acitretin, isotretinoin and/or tretinoin, in each case for the treatment of nail psoriasis, anti-infective drugs, e.g. clindamycin and/or erythromycin for the treatment of nail bed infection, preferably an antimycotic, so that it is suitable for the treatment of onychomycoses, in particular severe onychomycoses. The inventive formulation facilitates the permeation of sufficient active ingredient quantities through human fingernails and toenails, in particular of antimycotics, in order to also effectively treat severe onychomycoses. Correspondingly, the invention also provides the use of it.

With respect to the known prior art it is the object of the invention to provide an alternative formulation for active ingredients, in particular for antimycotic active ingredients, which in the case of topical application promotes their penetration through human fingernails and toenails, e.g. for the treatment of nail diseases and/or nail bed diseases, in particular of onychomycoses.

The pharmaceutical active ingredient is in particular an antimycotic active ingredient, e.g., terbinafine (base) or its water-soluble salt terbinafine hydrochloride, for the treatment of onychomycoses, in particular of severe onychomycoses. For the treatment of nail psoriasis (psoriasis of the nail) the pharmaceutical active ingredient is e.g. selected from the group comprising dithranol, preferably in combination with urea, vitamin D preparations, e.g., calcipotriol and/or tacalcitol (preferably in a concentration <100 μg/g formulation), corticoids, e.g. betamethasone, hydrocortisone, clobetasol, triamcinolonacetonide, prednicarbate and/or fluticasone (in total preferably in a concentration <1 percent by weight in the formulation), retinoids, e.g., adapalene, tazarotene, acitretin, isotretinoin and/or tretinoin (in total preferably in a concentration <1 percent by weight in the formulation). For the treatment of nail bed infection the pharmaceutical active ingredient is e.g. selected from the group comprising anti-infective drugs, e.g., clindamycin and/or erythromycin.

Preferably the inventive formulation contains or consists of 20 percent by weight poloxamer 407 (Lutrol F127), 12.5 percent by weight dimethyl isosorbide, 12.5 percent by weight isopropanol, 5 percent by weight medium chain triglycerides, 50 percent by weight water, bi-distilled, with a pharmaceutical active ingredient which is preferably an antimycotic, in particular from 1 to 10 percent by weight in the aforementioned formulation.

Surprisingly, it turns out that the formulation is also suitable for use in the treatment of onychomycoses if the pharmaceutical active ingredient is polar, amphiphilic or lipophilic. This is because the formulation facilitates a high penetration or permeation also in the case of polar or lipophilic pharmaceutical active ingredients through the human nail, e.g. of lipophilic antimycotic active ingredient, in particular terbinafine (base), and of polar antimycotic active ingredient, in particular terbinafine hydrochloride.

Correspondingly, the present invention provides the use of the aforementioned formulation in admixture with at least one pharmaceutical active ingredient, in particular an antimycotic, for use in the production of a pharmaceutical for use in the treatment of nail mycoses, in particular for topical application on the nail.

The conventional therapy of mycoses of the skin presently provides for the topical application of Lamisil-Dermgel (Novartis), which contains 1% terbinafine (base) as an active ingredient, and exhibits the following composition:

96% ethanol, isopropylmyristate, polysorbate 20, carbomer, sorbitan laurate, benzyl alcohol, sodium hydroxide, butyl hydroxyl toluene, purified water.

Surprisingly, it turns out that the inventive formulation causes a significantly higher permeation of antimycotic active ingredient, in particular terbinafine, which for example can be contained as a hydrochloride, through the nail, and therefore is especially suitable for use for the treatment of nail and/or nail bed diseases, which in particular are onychomycoses, through topical application on the nail.

In particular, the inventive formulation is suitable for the treatment of severe nail mycoses, since the permeation through the nail is so high that even severe nail mycoses can be treated. This is in contrast to previous therapies, in which case the topical application, for example of Lamisil, is restricted to application on the skin, while the antimycotic for weaker nail mycoses is used in a formulation as nail lacquer, however in the case of severe nail mycoses a systemic administration of the antimycotic takes place, in particular of terbinafine. This is because the formulations of terbinafine employed up to now for the therapy of nail mycoses have not yielded a sufficiently high permeation through the nail in order to effectively treat severe nail mycoses.

Along with the penetration reinforcing effect through nails, the inventive formulation has the advantage of exhibiting a significantly higher consistency at room temperature, e.g., solid or semisolid, than is the case of low temperatures, where it is for example liquid at 5-12° C. This gelling at higher temperatures, for the purposes of the invention also referred to as thermoreversible gelling, is especially advantageous in combination with the penetration reinforcing effect for pharmaceutical active ingredients, e.g. for antimycotic active ingredients, through the nail, since a simple blending of the active ingredient into the formulation at lower temperatures is possible, while at higher temperatures, in particular at body temperature, the higher consistency of the formulation promotes the continuance of the formulation on the site of application, i.e. on the nail, and therefore reinforces the continuous permeation of active ingredient through the nail, in particular as opposed to conventional ointment formulations, which become inviscid at body temperature and run from the site of application, and are absorbed by a dressing and are no longer available for permeation, respectively. Preferably the inventive formulation is therefore significantly less viscous at lower temperatures, e.g. at 5 to 10° C., for example inviscid, than in the case of temperatures higher by 10 to 20° C., e.g. at 15 to 30° C., at which the formulation is significantly more viscous, e.g. cream-like, semisolid or solid.

The inventive formulation preferably does not contain any adhesion improver, no film-former, no tenside and also no keratolytic agent.

The formulation according to the invention contains a polyether polyol in the range of 10-50 percent by weight, preferably 15-25 percent by weight, especially preferred 20 percent by weight. The polyether polyol can be selected from polyoxyethylene glycols having a molecular weight in the range of 1000 to 30,000, polyoxypropylene glycols in the range of 1000 to 30.000, polyoxyethylene-polyoxypropylene-block copolymers with a molecular weight in the range of 1000 to 30,000, and mixtures thereof. Polyethylene glycols can also be used as polyoxyethylene glycol, preferably for example polyethylene glycol 600 (PEG-12), polyethylene glycol 400 (PEG-8), and polyethylene glycol 300 (PEG-6).

Especially preferred polyether polyols are poloxamers having a molecular weight in the range of circa 2000 to 20,000 with a percentage of oxyethylene groups in the range of 40 to 90 percent by weight, for example poloxamer 124, poloxamer 188, poloxamer 237, and poloxamer 338, as defined in Ph. Eur. Poloxamer 407 is an especially preferred polyether polyol.

In addition, the inventive formulation contains dimethyl isosorbide in the range of 5-25 percent by weight, preferably in the range of 10-20 by weight or up to 15 percent by weight, especially preferred 12-13 percent by weight.

A further component of the inventive formulation is lipids, selected from medium chain triglycerides, fatty oils, paraffin oil, fluid waxes, isopropyl myristate, cetyloleate and mixtures thereof in the range of 2.5-10 percent by weight, more preferably 4-6 percent by weight. Medium chain triglycerides are especially preferred, for example in accordance with the definition of the European Pharmacopoeia (Ph. Eur., 4^{th} Edition, Main Edition 2002), i.e. a mixture of triglycerides of saturated fatty acids, mainly caprylic acid and capric acid, which exhibit at least 95% saturated fatty acids with 8 to 10 carbon atoms. Especially preferred is a fatty acid fraction of the following composition: a maximum of 2% caproic acid, 50 to 80% caprylic acid, 20 to 50% capric acid, a maximum of 3% lauric acid and a maximum of 1% myristic acid.

Further, the inventive formulation contains circa 5-20 percent by weight, preferably circa 10-15 percent by weight, especially preferred circa 12-13 percent by weight of a C₂- to C₄-alcohol, preferably ethanol, propanol, butanol, tert. butanol, especially preferred isopropanol.

The inventive composition additionally contains circa 40-60 percent by weight water, preferably circa 45-55 percent by weight water, especially preferably circa 50 percent by weight water.

The formulation according to the invention can contain one or more antimycotics as active ingredient, in particular amphiphilic, zwitterionic and strongly polar antimycotics. According to the invention, a preferred active ingredient is terbinafine, in particular its hydrochloride, in a percentage of circa 0.1-20 percent by weight, especially preferred circa 5-15 percent by weight, most preferably circa 10 percent by weight of the total composition.

The production process is based on the mixing of the formulation components, wherein advantageously no pre-treatment steps of individual components, for example heating up for liquefaction, are necessary.

Thus the polyether polyol can be provided as a solid, to which then dimethyl isosorbide (liquid) is added and subsequently the lipid, for example medium chain triglycerides (liquid). Subsequently, the C₂- to C₄-alcohol (liquid) is added, finally water. The formulation according to the invention can be produced by means of simple mechanical mixing at room temperature, for example stirring with a mechanical high-speed stirrer, e.g. an Unguator®, whereat the mixture gels directly during the stirring operation.

The mixed gelled preparation is for example storage stable over night or longer at room temperature. Pharmaceutical active ingredients can be incorporated by stirring. Suitable antimycotics are azole antimycotics, in particular bifonazole, clotrimazole, and econazole, squalene oxidase inhibitor antimycotics, in particular naftitine, morpholine derivative antimycotics, in particular amorolfine, as well as terbinafine, nystatin, griseofulvin, flucytosine, ciclopirox and mixtures thereof.

The production process according to the invention advantageously manages to manufacture the mixture with a simple high-speed mixer, for example an Unguator that can commonly be found at a pharmacy, so that the formulation can also be produced without additional need for equipment in quantities customary at pharmacies. The simple manufacturing method of the invention also offers advantages in large scale technical production, since no components have to to be specifically pre-treated, for example liquefaction by heating is dispensible.

As an alternative, the process for production can have the steps of joining of the formulation components polyether polyol, dimethyl isosorbide, C₂-C₄-alcohol and water, thus without lipid or an active ingredient, in a container and for example can be carried out in a Fanta dish. In the case of storage at a lower temperature, e.g. 4° C. over night, a clear viscous solution results. To this solution, preferably after heating to room temperature, the lipid portion can be added and be incorporated by stirring. The mixture becomes cloudy and in the case of additional heating, e.g., by the stirring itself, the mixture hardens to a thermoreversible gel. The active ingredient can then be incorporated into this gel by stirring.

The formulation according to the invention is shelf-stable and therefore it can advantageously be produced separately from the active ingredient to be incorporated later and can be stored without cooling, e.g. above 13° C. Due to the thermoreversible gelling of the formulation according to the invention, the incorporation of active ingredients is easy to handle, since the active ingredients are to be integrated into the formulation through simple mechanical stirring. If desired, the viscosity of the inventive formulation can be lowered by cooling, for example to temperatures of 5-12° C. By means of heating up to room temperature the formulation containing active ingredient that has been blended in, the desired viscosity of cream-like or semisolid to solid is achieved, without additional processing steps being necessary. This increase in viscosity at higher temperatures is dependent on the type and percentage of the active ingredient that has been blended in, wherein the temperature-dependent viscosity increase of the formulation for a blended-in active ingredient can be determined specifically both for the compound and for its quantity.

The formulation is suitable both for use in the treatment of nail diseases and nail bed diseases of humans and for the treatment of nail diseases and nail bed diseases of animals, e.g., of cloven hoofed animals and odd-toed ungulates, in particular in cattle, sheep, goats, horses, pigs, camels, elephants, hippopotamuses, dogs or cats. One preferred nail disease to be treated is a nail mycosis.

### Example 1

### Production of the Formulation of the Invention with Antimycotic

The following components are weighed in at room temperature into a conventional Unguator jar common in pharmacies as a mixing vessel:

20.0 g poloxamer 407 (Lutrol F 127, Bayer), 12.5 g dimethyl isosorbide (liquid), 5.0 g medium chain triglycerides (liquid, in accordance with European Pharmacopoeia, Ph. Eur.), 12.5 g isopropanol, and finally 50.0 g water with terbinafine hydrochloride to 1 percent by weight of the entire mixture.

A mechanical stirrer is inserted through the opening of the jar and the composition is mixed by means of stirring with an Unguator® at 5° C. The initially liquid mixture gels immediately during the stirring at circa 1450 rpm for 1.5 min.

The finished mixed, gel-like preparation is stored over night at room temperature.

The pH of the solution and of the gel-like mixture, respectively, can be set to any desired value.

### Example 2

### Measurement of the Permeation of Active Ingredient From the Formulation According to the Invention Through Nails

Bovine hoof discs of 100 μm thickness were used as examples for a human fingernail or toenail. The flux through the nail model was measured in the following way:

Bovine hoof discs were prepared by cutting out rectangular pieces of circa 2×2 cm from the soles of the hooves, equilibration of the cut-out pieces over night in water, cutting into disks of circa 100 μm thickness with the microtome and punching out of sections of circa 15 mm diameter from the discs and subsequent equilibration in PBS for 10 hours. In modified Franz diffusion cells, the diffusion was measured at 32° C.: As an acceptor, phosphate-buffer saline (PBS), pH 7.4, was provided in a measured volume of 5.2-6.3 mL. The equilibrated bovine hoof discs (permeation areas of 0.48-0.6 cm²) were fastened between the acceptor chamber and the donor chamber. Thermogel according to the invention or Lamisil was introduced into the donor chambers. The test suction was 100 μL in each case, and the acceptor was subsequently supplemented with fresh PBS. After a permeation for 40 hours at room temperature, the concentration of the terbinafine in the acceptor chamber was determined by means of HPLC (Zorbax column SB-C18, Agilent; mobile solvent: methanol:water 95:5 with 10 mM triethylamine at room temperature, isocratic; flow rate 1.5 mL/min, detection at 254 nm, 40 μL injected sample volume).

FIG. 1 shows the results, from which it is clear that the formulation according to the invention yields a significantly higher permation through nails than the comparative composition Dermgel.

The following data for the antimycotic were determined from the measured data:

| | Antimycotic | | | |
|---|---|---|---|---|
| | | | Total quantity | |
| | | accumulated | terbinafine | |
| | | quantity/area in | permeated | |
| | Flux | hooves after 40 h | and penetrated | |
| Formulation | (g/(cm² s)) | (μg/cm²) | (μg) | Delay time (min) |
| Thermogel | 6.59 × 10⁻¹⁰ +/− | 20.49 +/− 1.48 | 65.44 +/− 8.73 | 222.26 +/− 45.24 |
| according to the | 1.59 × 10⁻¹⁰ | | | |
| invention (n = 5) | | | | |
| Lamisil | 3.54 × 10⁻¹¹ +/− | 5.65 +/− 0.93 | 5.78 +/− 0.94 | 485.54 +/− 291.53 |
| Dermgel 1% | 1.67 × 10⁻¹¹ | | | |

On the example of an antimycotic active ingredient these data show that the flux of pharmaceutical active ingredient through the nail with the formulation of the invention is significantly higher, and the delay time (lag) is considerable shorter, so that with the formulation according to the invention, more pharmaceutical active ingredient is more rapidly transported through nails, and in the case of topical application on the nail results in an effective treatment of nail diseases and nail bed diseases, in particular of nail mycoses including severe nail mycoses.

Also, the quantity of the antimycotic accumulated in the nail model used as an example for an active ingredient, and the total quantity of the antimycotic which penetrated or permeated into and through the nail model show the considerably higher effective concentrations of the exemplary active ingredient antimycotic, which are introduced by means of the inventive formulation.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. Formulation for the treatment of nail and nail bed diseases, the formulation comprising:
0-20 wt. percent at least one of apolar, lipophilic, amphiphilic, zwitterionic or strongly polar pharmaceutical active ingredient,
10-50 wt. percent of a polyetherpolyol or a mixture thereof, selected from polyethylene glycol (α-hydro-ω-hydroxy-poly(oxy-1,2-ethane diol)), polypropylene glycol and/or poloxamer,
5-25% by weight dimethyl isosorbide,
2.5-10% by weight triglyceride, preferably medium-chain triglyceride, in particular according to the German Pharmacopoeia, and
5-20% by weight of a C1- to C4-alcohol or a mixture thereof,
remainder inactive liquid.

2. Formulation according to claim 1, wherein the active ingredient is an antifungal ingredient, and the nail bed or nail bed disease is onychomycosis.

3. Formulation according to claim 2, wherein the antifungal ingredient is azole antimycotics, squalene epoxidase inhibitors-antimycotics, morpholine derivative-antimycotics, terbinafine, nystatin, griseofulvin, flucytosine, and/or ciclopirox.

4. Formulation according to claim 1, wherein the ingredient is selected from a group that includes an active ingredient for nail psoriasis and the nail or nail bed disease is nail psoriasis.

5. Formulation according to claim 1, wherein the ingredient is selected from a group that includes an active ingredient for nail bed infections and the nail or nail bed disease is a nail bed infection.

6. Formulation according to claim 1, wherein the formulation consists of the ingredients listed in claim 1 and the inactive liquid is water, colorant and/or perfume.

7. Formulation according to claim 1, wherein the alcohol or mixture is ethanol and/or isopropanol

8. Formulation according to claim 1, wherein the nail or nail bed disease occurs on human or animals.

9. Formulation according to claim 1, wherein the formulation has no ibuprofen.

10. Formulation according to claim 1, wherein the poloxamer comprises poloxamer 407.

11. A method for treatment of nail and nail bed diseases, the method comprising:
providing a formulation, the formulation comprising
0-20 wt. percent at least one of apolar, lipophilic, amphiphilic, zwitterionic or strongly polar pharmaceutical active ingredient,
10-50 wt. percent of a polyetherpolyol or a mixture thereof, selected from polyethylene glycol (α-hydro-ω-hydroxy-poly(oxy-1,2-ethane diol)), polypropylene glycol and/or poloxamer,
5-25% by weight dimethyl isosorbide,
2.5-10% by weight triglyceride, preferably medium-chain triglyceride, in particular according to the German Pharmacopoeia, and
5-20% by weight of a C1- to C4-alcohol or a mixture thereof,
remainder inactive liquid, and
administering the formulation to the nail or nail bed region

12. Method according to claim 11, wherein the active ingredient is an antifungal ingredient, and the nail bed or nail bed disease is onychomycosis.

13. Method according to claim 12, wherein the antifungal ingredient is azole antimycotics, squalene epoxidase inhibitors-antimycotics, morpholine derivative-antimycotics, terbinafine, nystatin, griseofulvin, flucytosine, and/or ciclopirox.

14. Method according to claim 11, wherein the ingredient is selected from a group that includes an active ingredient for nail psoriasis and the nail or nail bed disease is nail psoriasis.

15. Method according to claim 11, wherein the ingredient is selected from a group that includes an active ingredient for nail bed infections and the nail or nail bed disease is a nail bed infection.

16. Method according to claim 11, wherein the formulation consists of the ingredients listed in claim 1 and the inactive liquid is water, colorant and/or perfume.

17. Method according to claim 11, wherein the alcohol or mixture is ethanol and/or isopropanol

18. Method according to claim 11, wherein the nail or nail bed disease occurs on human or animals.

19. Method according to claim 11, wherein said administering consists of topical application on the nail.

20. Method according to claim 11, wherein the formulation has no ibuprofen.

21. The method of claim 11, wherein the poloxamer comprises poloxamer 407.
